(19) United States
(12) Patent Application Publication
(10) Pub. No.: US 2026/0315126 A1
(43) Pub. Date: Oct. 15, 2026

(54) AUTOMATIC SCREW-PLACEMENT SYSTEM AND AN ORTHOPEDIC SURGICAL ROBOT

(71) Applicant: TINAVI MEDICAL TECHNOLOGIES CO., LTD., Beijing (CN)

(72) Inventors: Yue ZHOU, Beijing (CN); Wenzhao LI, Beijing (CN); Shengfeng ZHANG, Beijing (CN)

(73) Assignee: TINAVI MEDICAL TECHNOLOGIES CO., LTD., Beijing (CN)

(21) Appl. No.: 19/464,052

(22) Filed: Jan. 29, 2026

Related U.S. Application Data
(63) Continuation of application No. PCT/CN2025/090810, filed on Apr. 24, 2025.

(30) Foreign Application Priority Data
Mar. 18, 2025 (CN) 202520470415.9
Apr. 8, 2025 (CN) 202520642442.X

(51) Int. Cl. A61B 17/88; A61B 17/84; A61B 17/86; A61B 34/30

(52) U.S. Cl. CPC A61B 17/88; A61B 17/86; A61B 34/30; A61B 17/848

(57) ABSTRACT

An automatic screw-placement system comprises: a Kirschner wire, comprising a needle body and a needle head, the needle body being fixedly connected to the needle head, the needle head comprising a needle tip, the needle tip being located at the end of the needle head; and a pedicle screw, comprising a screw body and a milling assembly; a power platform, comprising a pin-insertion module and a screw-insertion module, the pin-insertion module being configured for inserting the K-wire into the patient's spine, and the screw-insertion module being configured to insert the pedicle screw into the pedicle of the patient's spine. Through the coordinated design of the pin-insertion module and the screw-insertion module, the Kirschner-wire positioning and screw-implantation processes are integrated into a platform, full automation of the entire process from the insertion of the Kirschner wire to the insertion of the pedicle screw is achieved.

### CROSS-REFERENCE TO RELATED APPLICATIONS

This application is a continuation of International Patent Application No. PCT/CN2025/090810 with a filing date of Apr. 24, 2025, designating the United States, now pending, and further claims priorities to Chinese Patent Application No. 202520470415.9 with a filing date of Mar. 18, 2025 and Chinese Patent Application No. 202520642442.X with a filing date of Apr. 8, 2025. The content of the aforementioned applications, including any intervening amendments thereto, is incorporated herein by reference.

### TECHNICAL FIELD

The present disclosure relates to the technical field of robotic automated pin placement, and specifically to a screw-placement system and an orthopedic surgical robot.

### BACKGROUND ART

In recent years, robot-assisted minimally invasive spinal surgery has been gradually adopted in clinical practice. Through the high-precision positioning of robotic arms, such systems can assist surgeons in the initial insertion of Kirschner wires (K-wires). However, existing technologies still have the following limitations:

First, current systems can only achieve the initial fixation of the K-wire, while the implantation of the pedicle screw still relies on the surgeon to manually advance the screw along the K-wire trajectory or to use traditional tools. This process requires repeated adjustments of the screw's position and angle, is time-consuming, and is prone to human error, which can reduce screw placement accuracy—particularly in cases involving complex anatomical structures, where risks are further elevated.

Second, existing screw-insertion tools often lack sufficient driving force and rotational speed, making it difficult to efficiently grind through the dense cortical bone at the pedicle entry point. This can easily lead to K-wire slippage, screw deviation, and other issues. For patients with abnormal bone density (such as sclerosis or osteoporosis), traditional tools lack adaptive control, which may result in inadequate preparation of the bone channel or even cause damage to surrounding tissues.

Furthermore, in existing devices, the K-wire insertion module and the screw-implantation module are typically independent structures. The workflow requires multiple instrument changes, which not only prolongs the operation time but also increases the risk of intraoperative contamination. Therefore, achieving full-process automation—from precise K-wire placement to automatic screw insertion—while improving the efficiency and stability of bone-channel preparation has become an urgent technical challenge.

Accordingly, the existing technology still requires further development.

### SUMMARY OF PRESENT INVENTION

The purpose of the present disclosure is to overcome the above technical deficiencies and provide an automatic screw-placement system and an orthopedic surgical robot, so as to solve the problems existing in the prior art.

To achieve the above technical purpose, according to a first aspect of the present disclosure, the disclosure provides an automatic screw-placement system, comprising:

A Kirschner wire (K-wire), comprising a needle body and a needle head, the needle body being fixedly connected to the needle head, the needle comprising a needle tip, the needle tip being located at an end of the needle head;

A pedicle screw, comprising a screw body and a milling assembly; and

A power platform, comprising a pin-insertion module and a screw-insertion module, the pin-insertion module being configured to insert the K-wire into the patient's spine, and the screw-insertion module being configured to insert the pedicle screw into a pedicle of the patient's spine.

Specifically, the pin-insertion module is detachably connected to the K-wire, the screw-insertion module comprises a robotic arm interface, and the robotic arm interface is configured to connect to a robotic arm.

Specifically, the power platform further comprises a screw-mounting module, and the pedicle screw is mounted on the screw-mounting module; the screw-insertion module is fixedly connected to the pin-insertion module, and the screw-mounting module is detachably connected to the screw-insertion module.

Specifically, a diameter of the needle body of the K-wire is greater than a diameter of the needle tip, the end of the needle tip includes a double-sided cutting edge and a chip-removal groove, the double-sided cutting edge is configured to cut bone, and the chip-removal groove is configured to remove debris.

Specifically, the pin-insertion module comprises a transmission shaft, and the screw-insertion module comprises a screw-insertion housing, and a first motor and a main gear are disposed in the screw-insertion housing. The first motor is connected to the main gear, the main gear is connected to the transmission shaft, and the first motor is configured to drive the main gear to rotate and thereby rotate the driving shaft.

Specifically, a second motor and a gear shaft are further disposed inside the screw-insertion housing, the second motor is connected to the gear shaft, the second motor is configured to drive the gear shaft to rotate, and an inner sleeve is disposed inside the second motor for allowing the Kirschner wire to pass through.

Specifically, a third motor, a reducer, and a clutch are further disposed inside the screw-insertion housing, the third motor is connected to the reducer, the reducer is connected to the clutch. One end of the gear shaft is provided with a driving gear, the driving gear is connected to the clutch, and the other end of the gear shaft is connected to a first connecting device, the first connecting device is detachably connected to the screw-mounting module.

Specifically, the pin-insertion module comprises a pin-insertion housing, and an upper sleeve and a fixing sleeve are disposed inside the pin-insertion housing. The upper sleeve and the fixing sleeve are fixedly connected, and the internal spaces of the upper sleeve and the fixing sleeve communicate with each other for allowing the Kirschner wire to pass through, and the fixing sleeve is fixedly connected to the transmission shaft.

Specifically, the power platform further comprises a retaining frame, the retaining frame is fixedly connected to the screw-insertion module, and the retaining frame is detachably connected to the screw-mounting module.

Specifically, a movement space extending along an extension direction of the screw body is defined inside the screw body, the movement space is configured for the K-wire to pass through. The milling assembly is mounted at an end of the screw body, threads are defined on a surface of the milling assembly, and grooves are formed on the threads. The milling assembly comprises at least two cutting edges, and the cutting edges are circumferentially spaced apart at the end of the screw body.

Specifically, the pedicle screw further comprises a screw head, the screw head is disposed at the end of the screw body away from the milling assembly. A mounting hole is formed in the screw head, and the mounting hole communicates with the movement space.

Specifically, the pedicle screw further comprises a screw-body long tail, the screw-body long tail is fixedly connected to the screw head; the screw-body long tail has a connecting end and a fixing end, a first mounting space is defined in the connecting end of the screw-body long tail, a second mounting space is defined in the fixing end of the screw-body long tail, the first mounting space is communicated with the second mounting space, and the screw head is movably disposed inside the first mounting space.

Specifically, the pedicle screw further comprises a connecting assembly, the connecting assembly comprises a first connecting portion and a second connecting portion connected in sequence. The first connecting portion and the second connecting portion both extend in the extension direction of the screw body. The first connecting portion is connected to the screw-mounting module, the second connecting portion is rotatably disposed in the second mounting space, and the second connecting portion is rotatably connected to the fixing end of the screw-body long tail.

According to a second aspect of the present disclosure, the disclosure provides an orthopedic surgical robot, comprising:
the above automatic screw-placement system;
a robotic system, comprising a main control cart and a main machine cart, the main control cart comprising an industrial computer, a display, and an optical camera, the main machine cart comprising a robotic arm, the end of the robotic arm comprising a first connecting structure configured to connect the power platform; and
an optical marker module fixedly disposed on the outer surface of the first connecting structure, and configured to identify the pose of the robotic arm.

### Beneficial Effects

The present disclosure provides an automatic screw-placement system and an orthopedic surgical robot. The orthopedic surgical robot includes a robot system, a power platform, and corresponding Kirschner wires and pedicle screws. The power platform is fixedly connected to the end of the robotic arm and is rigidly connected to the optical marker module. Through the coordinated design of the pin-insertion module and the screw-insertion module, the automatic pin placement system integrates the K-wire positioning and screw implantation processes into a single robotic-arm operation platform. In combination with command control from the robot system, the system significantly shortens the operation time, further reduces surgical risks, solves the technical problems in the prior art such as K-wire slippage and reliance on manual screw insertion, and achieves full-process automation and precise control from K-wire positioning to screw implantation, thus providing a more efficient, stable, and safe solution for robot-assisted spinal surgery.

### DESCRIPTION OF THE DRAWINGS

FIG. 1 is a schematic diagram showing an automatic screw-placement system according to the specific embodiment of the present disclosure;

FIG. 2 is a schematic diagram showing an orthopedic surgical robot according to the specific embodiment of the present disclosure;

FIG. 3 is a schematic diagram showing working process of a main machine cart according to the specific embodiment of the present disclosure;

FIG. 4 is a structural schematic diagram showing a Kirschner wire according to the specific embodiment of the present disclosure;

FIG. 5 is a plan view of the Kirschner wire according to the specific embodiment of the present disclosure;

FIG. 6 is a 3D view of the Kirschner wire according to the specific embodiment of the present disclosure;

FIG. 7 is a schematic diagram showing inserting the Kirschner wire into the pedicle according to the specific embodiment of the present disclosure;

FIG. 8 is a schematic diagram showing a grinding channel of the Kirschner wire according to the specific embodiment of the present disclosure;

FIG. 9 is a structural schematic diagram showing a screw-mounting module according to the specific embodiment of the present disclosure;

FIG. 10 is a structural schematic diagram showing a pedicle screw according to the specific embodiment of the present disclosure;

FIG. 11 is a structural schematic diagram showing a milling assembly comprising six cutting teeth according to the specific embodiment of the present disclosure;

FIG. 12 is a structural schematic diagram showing the milling assembly comprising two cutting teeth according to the specific embodiment of the present disclosure;

FIG. 13 is a structural schematic diagram showing a compression ring according to the specific embodiment of the present disclosure;

FIG. 14 is a structural schematic diagram showing a connecting assembly according to the specific embodiment of the present disclosure;

FIG. 15 is a sectional schematic diagram showing a screw-body long tail according to the specific embodiment of the present disclosure;

FIG. 16 is a sectional view showing an automatic screw-placement system according to the specific embodiment of the present disclosure;

FIG. 17 is a structural sectional view showing a screw-insertion module according to the specific embodiment of the present disclosure;

FIG. 18 is a top view of the gear shaft provided in the specific embodiment of the present disclosure;

FIG. 19 is a partial front view showing a gear shaft according to the specific embodiment of the present disclosure;

FIG. 20 is a structural sectional view showing a screw-insertion module according to the specific embodiment of the present disclosure;

FIG. 21 is a structural schematic diagram showing a transmission shaft according to the specific embodiment of the present disclosure;

FIG. 22 is a structural schematic diagram showing a fixing sleeve according to the specific embodiment of the present disclosure.

The above drawings include the following reference numerals:
1, Kirschner wire; 2, pin-insertion module; 3, screw-insertion module; 4, needle body; 5, needle head; 6, needle tip; 7, pedicle screw; 8, robotic arm interface; 10, screw-mounting module; 12, double-sided cutting edge; 13, chip-removal groove; 14, transmission shaft; 15, screw-insertion housing; 16, first motor; 17, main gear; 18, second motor; 19, gear shaft; 20, inner sleeve; 21, third motor; 22, reducer; 23, clutch; 24, driving gear; 25, first connecting device; 26, pin-insertion housing; 27, upper sleeve; 28, fixing sleeve; 29, retaining frame; 30, screw body; 31, milling assembly; 33, movement space; 34, screw head; 36, screw-body long tail; 37, connecting end; 38, fixing end; 39, first mounting space; 40, second mounting space; 41, connecting assembly; 42, first connecting portion; 43, second connecting portion; 44, hexagonal shaft; 45, positioning groove; 46, fixing nut; 47, orientation ring; 48, fixing shaft; 49, ball groove; 50, compression ring; 51, rotation space; 52, through hole; 54, third connecting portion; 55, first gear; 56, first lead screw-nut; 57, second gear; 58, hexagonal anti-rotation structure; 60, screw external thread; 61, connecting thread; 62, main control cart; 63, main machine cart; 64, industrial computer; 65, display; 66, optical camera; 67, robotic arm; 68, optical marker module; 69, power platform; 70, patient tracker.

### DETAILED DESCRIPTION OF PREFERRED EMBODIMENTS

In order to enable those skilled in the art to better understand the technical solutions of the present disclosure, the technical solutions of the present disclosure are described clearly and completely in combination with the drawings of the disclosure. Based on the embodiments in this application, other similar embodiments obtained by a person of ordinary skill in the art without inventive effort shall also fall within the scope of protection of this application. In addition, the directional terms used in the following embodiments, such as “upper,” “lower,” “left,” “right,” etc., are only with reference to the direction of the drawings; therefore, the directional terms are intended for illustration and not to limit the scope of the disclosure. The present disclosure is further described below in combination with the drawings and preferred embodiments.

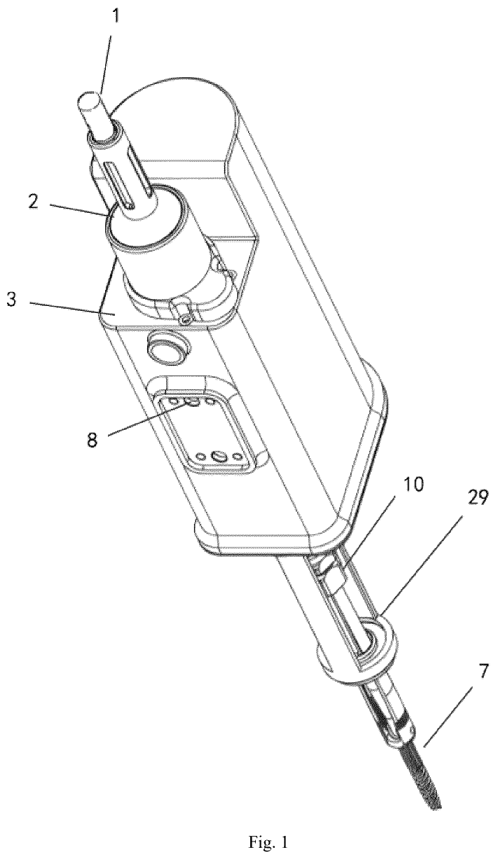

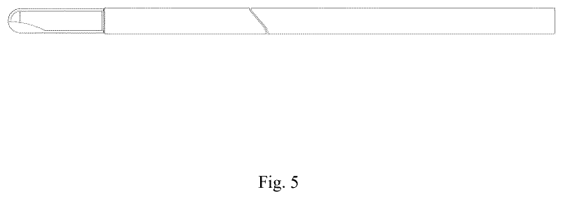

Referring to FIG. 1-FIG. 22, this embodiment provides an automatic screw-placement system, comprising: a Kirschner wire 1, a pedicle screw 7, and a power platform 69.

The Kirschner wire 1 includes a needle body 4 and a needle head 5, the needle body 4 is fixedly connected to the needle head 5, and the needle head 5 includes a needle tip 6, the needle tip 6 is located at an end of the needle head 5.

The pedicle screw 7 includes a screw body 30 and a milling assembly 31.

The power platform 69 includes a pin-insertion module 2 and a screw-insertion module 3, the pin-insertion module 2 is configured to insert the Kirschner wire 1 into the patient's spine, and the screw-insertion module 3 is configured to insert the pedicle screw 7 into the pedicle of the patient's spine.

It can be understood that the needle tip 6 of the Kirschner wire 1 in this embodiment is designed as hemispherical, rather than a conventional triangular or conical tip. During operation, the Kirschner wire 1 is inserted into the bone solely by rotation or slow advancement of the tip. The present disclosure employs a hemispherical needle tip 6 design, which, by increasing the contact area between the needle tip 6 and the bone surface and combining precise force application by the robotic arm, can effectively distribute puncture pressure, reduce the risk of Kirschner wire 1 slippage on hard bone surfaces, and prevent deviation of the bone channel trajectory caused by misalignment of the Kirschner wire 1, thereby providing a precise guiding basis for subsequent screw implantation. Moreover, by controlling the control module to operate the pin-insertion module 2 and screw-insertion module 3, automatic pin placement or screw insertion can be performed, solving technical problems in the prior art such as Kirschner wire 1 slippage and manual screw implantation reliance.

Referring to FIG. 1, in the automatic pin placement system of this embodiment, the pin-insertion module 2 is detachably connected to the Kirschner wire 1. The screw-insertion module 3 includes a robotic arm interface 8, which is configured to connect to the robotic arm 67. The power platform 69 further includes a screw-mounting module 10, on which the pedicle screw 7 is mounted. The screw-insertion module 3 is fixedly connected to the pin-insertion module 2, and the screw-mounting module 10 is detachably connected to the screw-insertion module 3. The fixed and detachable connections between modules ensure surgical precision while further optimizing the continuity and flexibility of the operation workflow.

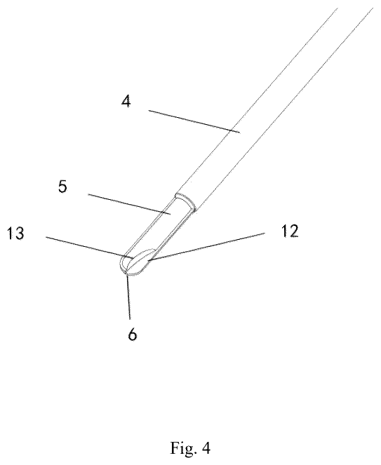

Referring to FIG. 4-FIG. 6, in the automatic pin placement system of this embodiment, the diameter of the needle body 4 is greater than that of the needle head 5, ensuring the stability of the Kirschner wire 1 after insertion. The end of the needle head 5 includes a double-sided cutting edge 12 and a chip-removal groove 13. The double-sided cutting edge 12 is configured to cut bone, and the chip-removal groove 13 is configured to remove debris. The double-sided cutting edge 12 is located at the end of the needle head 5 and presents a sharp edge, while the chip-removal groove 13 extends along the needle head 5 to ensure smooth removal of bone debris during cutting, preventing blockage and improving surgical efficiency. The double-sided cutting edge 12 and the chip-removal groove 13 are adjacent and jointly perform cutting and debris removal. The needle tip 6 is hemispherical, which reduces the risk of injury to nerves or other tissues if the Kirschner wire 1 accidentally penetrates the medullary cavity beyond the planned depth. The hemispherical needle head 5 serves as a buffer, avoiding severe damage to nerves or tissues within the medullary cavity, significantly improving surgical safety and reducing complications, while solving the problem of Kirschner wire slippage.

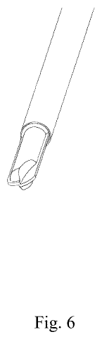

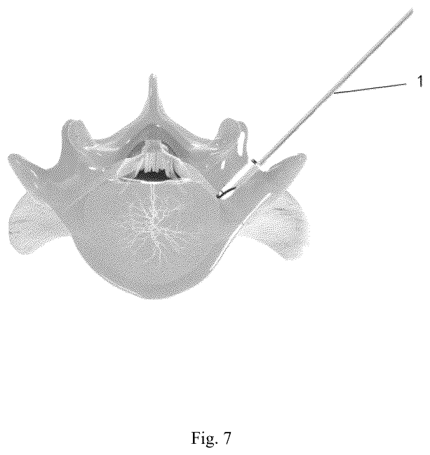

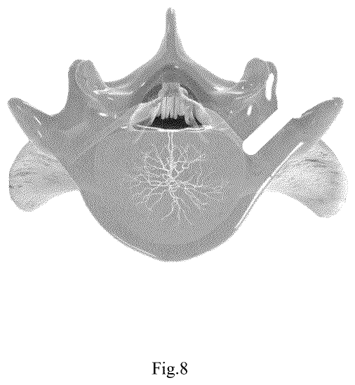

Further, referring to FIG. 7-FIG. 8, during operation, the Kirschner wire 1 is driven by the pin-insertion module 2 to rotate at high speed. Under the action of the double-sided cutting edge 12 of the needle head 5, the bone on the cutting channel remains within the pedicle, as shown in FIG. 6, guiding the subsequent insertion of the pedicle screw 7. The needle head 5 cuts bone and guides the Kirschner wire 1 into the bone. Its design allows the Kirschner wire 1 to cut the bone effectively at high speed and remove debris through the chip-removal groove 13. If the Kirschner wire 1 penetrates the medullary cavity beyond the planned depth, the hemispherical needle head 5 protects nerves or tissues within the medullary cavity, enabling high-speed grinding without slippage and safeguarding the patient from accidental injury.

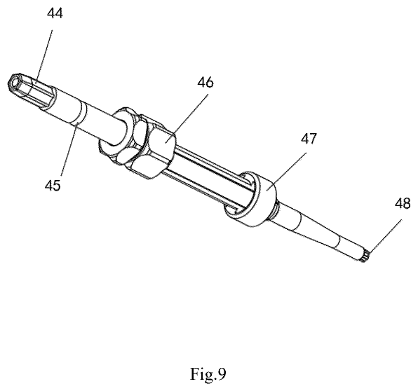

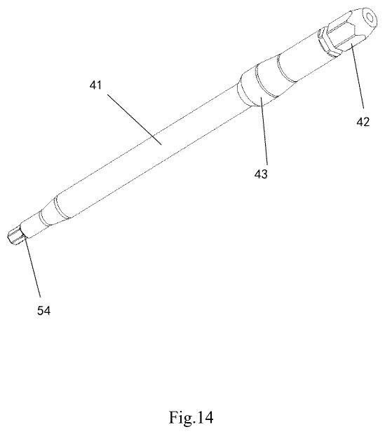

Referring to FIG. 9, in some specific embodiments, the pedicle screw 7 is mounted on the screw-mounting module 10. From top to bottom, the screw-mounting module 10 is sequentially provided with a hexagonal shaft 44, a positioning groove 45, a fixing nut 46, an orientation ring 47, and a fixing shaft 48. The hexagonal shaft 44 serves as the core component for power transmission, its hexagonal cross-section matches the interface of the screw-insertion module 3 and transmits rotational torque to the pedicle screw 7. The multi-surface contact of the hexagonal cross-section effectively prevents slippage during torque transmission and ensures the stability of the pedicle screw 7 rotation. The positioning groove 45 provides mechanical feedback and depth-limiting control for screw implantation. Referring to FIG. 14, a ball groove 49 is provided on the first connecting device 25 for mounting a limiting steel ball, i.e., a positioning ball. When the pedicle screw 7 reaches the predetermined depth, the positioning groove 45 cooperates with the positioning ball on the first connecting device 25 to ensure proper installation and locking of the screw-mounting module 10. The fixing nut 46 locks the connection between the pedicle screw 7 and the screw-mounting module 10 to prevent screw detachment during implantation. The orientation ring 47 controls the implantation angle and direction of the pedicle screw 7, ensuring consistency with preoperative planning. The fixing shaft 48 serves as the main supporting structure of the screw-mounting module 10, connecting components and transmitting axial pushing force evenly to the screw tip, avoiding localized stress-induced bone damage. This mechanical design addresses problems in traditional manual operations caused by insufficient torque, angle deviations, or depth loss, significantly improving surgical safety and efficiency.

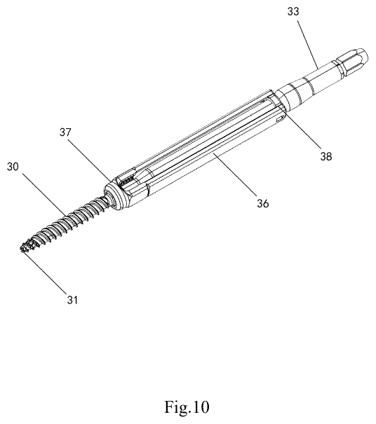

Referring to FIG. 10, in some specific embodiments, the pedicle screw 7 is detachably connected to the screw-mounting module 10. The pedicle screw 7 includes a screw body 30 and a milling assembly 31. A movement space 33 extending along the screw body 30 is provided within the screw body 30 for the Kirschner wire 1 to pass through. The milling assembly 31 is mounted at the end of the screw body 30. Threads are provided on the surface of the milling assembly 31, with grooves on the threads. The milling assembly 31 includes at least two cutting teeth, which are circumferentially spaced at the end of the screw body 30.

Further, when the Kirschner wire 1 needs to contact the target bone, the milling assembly 31 rotates at high speed to cut a recess in the target bone, preferably at a rotational speed of 5,000 RPM-15,000 RPM, allowing one end of the Kirschner wire 1 to move through the movement space 33 into the recess, guiding the implantation direction of the screw body 30.

Using the above technical solution, the threads on the milling assembly 31, with grooves on the threads, effectively cut bone and open a channel for the screw to enter. The grooves facilitate debris removal during cutting, preventing blockage and maintaining cutting efficiency. At least two cutting teeth are circumferentially spaced to increase cutting efficiency. Multiple teeth provide multi-point contact during rotation, enlarging the cutting area and improving cutting efficiency. This pedicle screw design comprehensively considers surgical precision, cutting efficiency, and fixation stability, effectively guiding and implanting the screw while efficiently cutting bone and ensuring secure fixation, providing reliable technical support for orthopedic surgery.

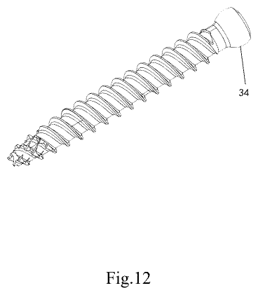

Referring to FIG. 12, in some specific embodiments, the milling assembly 31 may have two cutting teeth, each extending along a preset direction and positioned on opposite sides of the screw body 30.

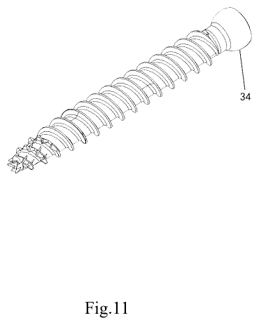

Referring to FIG. 11, in some embodiments, the milling assembly 31 may have six cutting teeth, each extending along a preset direction and circumferentially spaced at the end of the screw body 30. The selection of the number and layout of cutting teeth should be based on surgical requirements, bone hardness, and required cutting efficiency and precision.

Specifically, referring to FIG. 11 or FIG. 12, the pedicle screw 7 further includes a screw head 34, disposed at the end of the screw body 30 opposite the milling assembly 31. A mounting hole is formed in the screw head 34, communicating with the movement space 33.

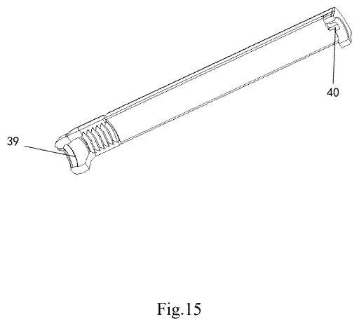

Specifically, referring to FIG. 14, FIG. 15, and FIG. 10, the pedicle screw 7 further includes a screw-body long tail 36, which is fixedly connected to the screw head 34. The screw-body long tail 36 has a connecting end 37 and a fixing end 38. A first mounting space 39 is formed at the connecting end 37, and a second mounting space 40 is formed at the fixing end 38. The first mounting space 39 communicates with the second mounting space 40, and the screw head 34 is movably disposed within the first mounting space 39. The design of the screw-body long tail 36 facilitates rapid implantation of the screw body 30 and increases implantation speed.

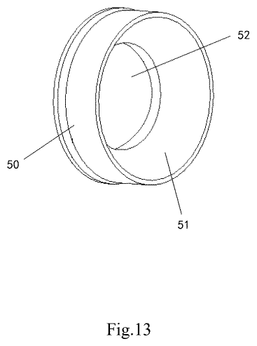

Referring to FIG. 13, the pedicle screw 7 further includes a retaining ring 50, which is disposed within the first mounting space 39. One side of the retaining ring 50 defines a rotation space 51 and a through-hole 52 communicating with the rotation space 51. The screw head 34 is movably disposed within the rotation space 51. The design of the retaining ring 50 facilitates limiting the movement of the screw head 34, preventing the screw head 34 from detaching from the screw-body long tail 36.

Specifically, referring to FIG. 14, the pedicle screw 7 further includes a connecting assembly 41, which comprises a first connecting part 42 and a second connecting part 43 connected in sequence. Both the first connecting part 42 and the second connecting part 43 extend along the longitudinal direction of the screw body 30. The first connecting part 42 is connected to the screw-mounting module 10. The second connecting part 43 is rotatably disposed within the second mounting space 40 and is rotatably connected to the fixing end 38 of the screw-body long tail 36.

Referring to FIG. 15, in some specific embodiments, the connecting assembly 41 has a first connecting end and a second connecting end. The first connecting end of the connecting assembly 41 is configured to pass through the second mounting space 40 and the through-hole 52 to connect to the screw head 34. The second connecting end of the connecting assembly 41 is for connection to the screw-mounting module 10. The screw-insertion module 3 drives the screw body 30 to rotate forward or backward through the connecting assembly 41.

In some specific embodiments, referring to FIG. 14, the connecting assembly 41 further includes a third connecting part 54 sequentially connected to the first connecting part 42 and the second connecting part 43. The first connecting part 42, second connecting part 43, and third connecting part 54 all extend along the longitudinal direction of the screw body 30. The first connecting part 42 is connected to the fixing shaft 48 of the screw-mounting module 10. The second connecting part 43 is rotatably disposed within the second mounting space 40 and rotatably connected to the fixing end 38 of the screw-body long tail 36. The third connecting part 54 sequentially passes through the second mounting space 40, the first mounting space 39, and the through-hole 52 to connect to the screw head 34. Through the first, second, and third connecting parts 42, 43, and 54, the screw-mounting module 10, the screw-body long tail 36, and the screw head 34 are connected, preparing the screw body 30 for implantation. This process does not require manual insertion, and robotic implantation achieves faster and more precise results.

Further, the screw-mounting module 10 can be replaced by other tools, such as an automatic retractor or other powered surgical instruments.

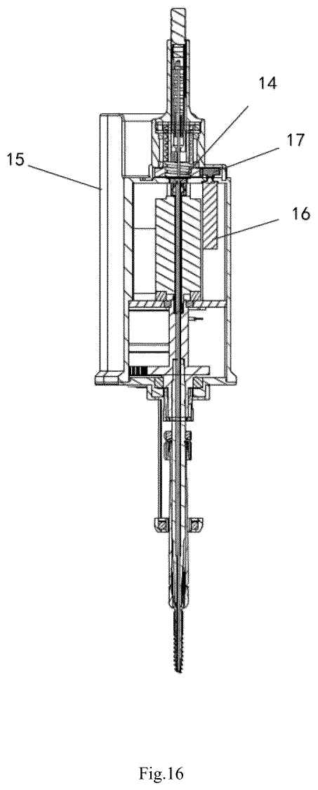

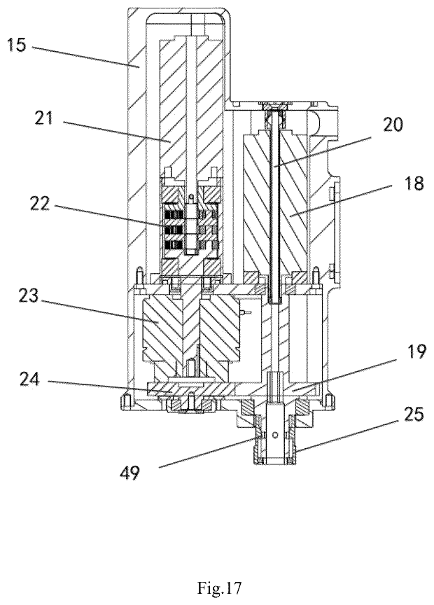

Referring to FIG. 16 and FIG. 17, in the automatic pin placement system of this embodiment, the pin-insertion module 2 includes a transmission shaft 14, and the screw-insertion module 3 includes a screw-insertion housing 15. Inside the screw-insertion housing 15, a first motor 16 and a main gear 17 are provided. The first motor 16 is connected to the main gear 17, and the main gear 17 is connected to the transmission shaft 14. The first motor 16 drives the main gear 17 to rotate, thereby driving the transmission shaft 14 to rotate.

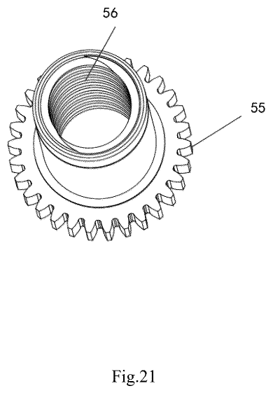

Further, referring to FIGS. 16 and 17, the first motor 16 includes an output shaft, and the main gear 17 is mounted on the output shaft of the first motor 16 via a key connection or other suitable fixing method (e.g., fastening screws), ensuring that the rotation of the output shaft of the first motor 16 is directly transmitted to the main gear 17. The main gear 17 is connected to the transmission shaft 14. As shown in FIG. 21, the transmission shaft 14 includes a first gear 55 and a first lead screw nut 56, so the main gear 17 and transmission shaft 14 are engaged through gear meshing to achieve transmission.

Specifically, referring to FIG. 16 and FIG. 17, the screw-insertion housing 15 further includes a second motor 18 and a gear shaft 19. The second motor 18 is connected to the gear shaft 19 and is configured to drive the gear shaft 19 to rotate. An inner sleeve 20 is provided inside the second motor 18 to allow the Kirschner wire 1 to pass through.

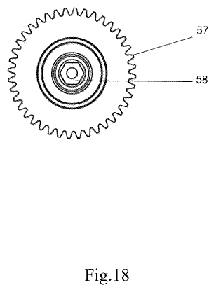

Referring to FIG. 17 and FIG. 18, in some specific embodiments, the gear shaft 19 includes a second gear 57 and a hexagonal anti-rotation structure 58, as shown in FIG. 15. The hexagonal anti-rotation structure 58 matches the hexagonal shaft 44 of the screw-mounting module 10. The hexagonal cross-section of the anti-rotation structure 58 forms a rigid fit with the corresponding interface of the connected component, ensuring that the gear shaft 19 rotates synchronously with adjacent components and preventing power loss caused by slippage or misalignment during torque transmission. The inner sleeve 20 is positioned at the center of the second motor 18, allowing the Kirschner wire 1 to pass stably during operation, avoiding interference while maintaining precise position control.

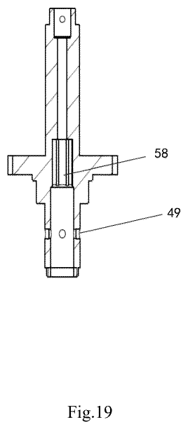

Further, the output shaft of the second motor 18 can be designed as a hexagonal shaft 44 that closely matches the inner hole of the hexagonal anti-rotation structure 58 of the gear shaft 19, as shown in FIG. 18 and FIG. 19. The hexagonal cross-section achieves zero-clearance engagement through multi-surface contact, ensuring no relative slip during torque transmission. The inner sleeve 20 of the second motor 18 passes through its output shaft center, and a coaxial through-hole is provided in the hexagonal anti-rotation structure 58 of the gear shaft 19, forming a continuous channel for the Kirschner wire 1.

Specifically, referring to FIG. 14, the screw-insertion housing 15 further includes a third motor 21, a reducer 22, and a clutch 23. The third motor 21 is connected to the reducer 22, and the reducer 22 is connected to the clutch 23.

Referring to FIG. 17, in some embodiments, the output shaft of the third motor 21 is directly connected to the input of the reducer 22, which can use a key connection or coupling to ensure efficient transmission of rotation. The output of the reducer 22 is connected to the input of the clutch 23, which can be achieved via a spline connection or rigid engagement between the hexagonal anti-rotation structure 58 and the clutch input. The reducer 22 converts the high-speed, low-torque output of the third motor 21 into low-speed, high-torque suitable for subsequent transmission, and the clutch 23 controls engagement and disengagement of the power.

Specifically, referring to FIG. 17, one end of the gear shaft 19 is equipped with a driving gear 24, which is connected to the clutch 23. The other end of the gear shaft 19 is connected to the first connecting device 25, which is detachably connected to the screw-mounting module 10.

Referring to FIG. 17, in some embodiments, the output of the clutch 23 is connected to the driving gear 24. During operation, when the clutch 23 engages, the driving gear 24 is driven to rotate. When the clutch 23 disengages, the driving gear 24 stops rotating. The connection between the clutch 23 and the driving gear 24 can be implemented by splines, keys, or other mechanical connections. The driving gear 24 is mounted on the gear shaft 19 and drives the gear shaft 19 to rotate through meshing. The other end of the gear shaft 19 is fixedly connected to the first connecting device 25, which facilitates convenient installation and removal of the screw-mounting module 10.

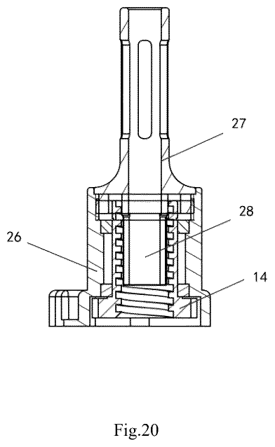

Referring to FIG. 20, the pin-insertion module 2 includes a pin-insertion housing 26. Inside the pin-insertion housing 26, an upper sleeve 27 and a fixing sleeve 28 are provided and fixedly connected. The fixing sleeve 28 is fixedly connected to the transmission shaft 14. This design ensures that the transmission shaft 14 drives the fixing sleeve 28 to move during rotation. The Kirschner wire 1 is detachably connected to the upper sleeve 27.

Specifically, referring to FIG. 20 and FIG. 21, the transmission shaft 14 includes a first gear 55 and a first lead screw nut 56. The first lead screw nut 56 has lead screw threads, and the fixing sleeve 28 is provided with screw external threads 60. The fixing sleeve 28 and transmission shaft 14 are connected via threaded engagement.

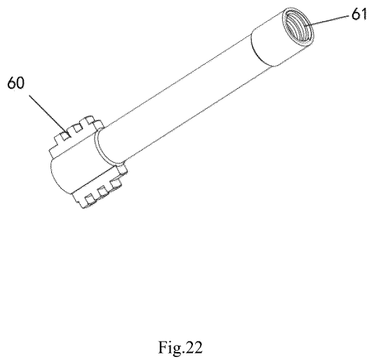

Referring to FIG. 20 and FIG. 22, the upper sleeve 27 is located above the fixing sleeve 28. A connecting thread 61 is provided above the fixing sleeve 28, and screw external threads 60 are provided below. The upper sleeve 27 and fixing sleeve 28 are fixed by threaded connection. The fixing sleeve 28 is also connected to the driving shaft 14 via threaded connection. A coaxial through-hole is provided inside the upper sleeve 27 and fixing sleeve 28 for the Kirschner wire 1, which together with the inner sleeve 20 in the screw-insertion module 3 forms a channel for the Kirschner wire 1. The transmission shaft 14 is connected with the main gear 17 via the first gear 55.

Referring to FIG. 1, the automatic pin placement system in this embodiment further includes a retaining frame 29, which is fixedly connected to the screw-insertion module 3 and detachably connected to the screw-mounting module 10. This helps stabilize high-speed rotation of the screw-mounting module 10, which in turn drives high-speed rotation of the pedicle screw 7.

It should be further noted that, referring to FIG. 1-FIG. 22, the working process of the automatic screw-placement system in this embodiment is as follows:

### (1) Preoperative Preparation

Before spinal surgery, the operation process for the Kirschner wire 1 and the screw-mounting module 10 comprises: first, installing the pedicle screw 7 on the screw-mounting module 10 via the fixing nut 46, then mounting the screw-mounting module 10 onto the screw-insertion module 3 via the first connecting device 25. Afterward, the assembly is mounted on the robotic arm through the robotic arm interface 8, completing the preoperative preparation.

### (2) Surgical Procedure

After the robotic arm drives the power platform 69 in this embodiment to the planned position, the robotic system issues a command to perform automatic screw placement. The first motor 16 in the screw-insertion module 3 drives the main gear 17 to rotate, causing the transmission shaft 14 to rotate. Under the action of the first lead screw nut 56 of the transmission shaft 14, the fixing sleeve 28 and the Kirschner wire 1 move downward to the required depth. Then, the second motor 18 drives the gear shaft 19 to rotate at high speed, causing the screw-mounting module 10 to rotate at high speed. The retaining frame 29 assists the screw-mounting module 10 in stabilizing high-speed rotation, while the robotic arm moves downward along the axis of the pedicle screw 7 until the milling component 31 of the pedicle screw 7 completely mills the cortical bone of the patient's spine. Afterward, the robotic arm and the first motor 16 stop moving. The third motor 21 rotates at low speed under the action of the reducer 22, and the clutch 23 engages, driving the driving gear 24 to rotate. The driving gear 24 drives the gear shaft 19 and the screw-mounting module 10 to rotate, thereby accomplishing the insertion of the pedicle screw 7.

It should be noted that this embodiment provides an automatic screw-placement system. Through the coordinated design of the pin-insertion module and screw-insertion module, the system integrates Kirschner wire positioning with the screw implantation workflow onto the same robotic arm platform. Together with the robotic system's command control, the system significantly shortens surgical time, further reduces the risk of contamination, addresses the technical issues in the prior art such as the Kirschner wire easily slipping and screw implantation relying on manual operation, and achieves fully automated and precise control from Kirschner wire positioning to screw insertion. This provides a more efficient, stable, and safe solution for robot-assisted spinal surgery.

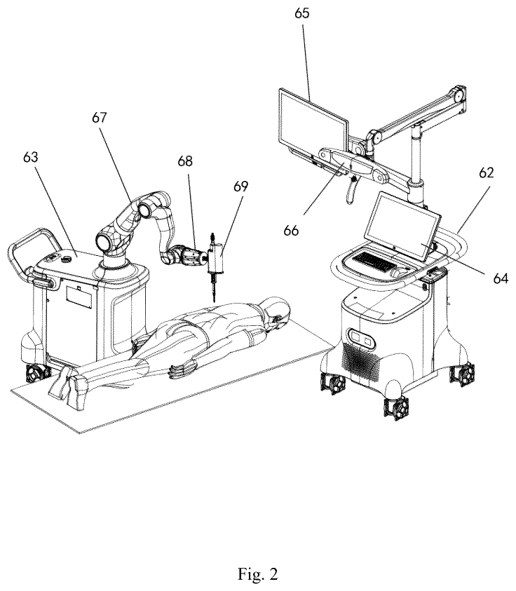

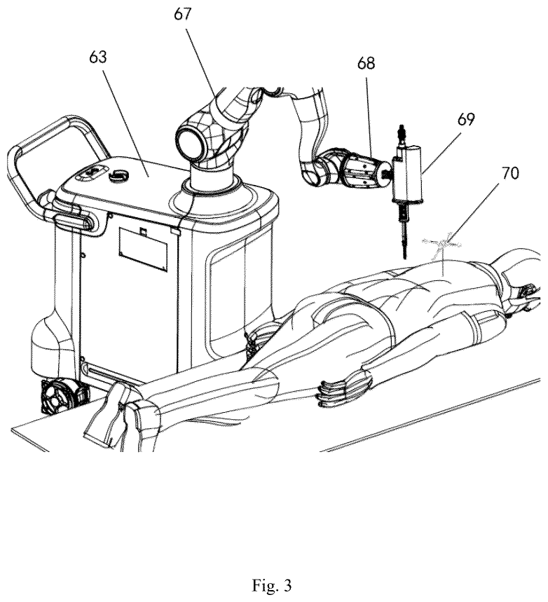

Referring to FIG. 2 and FIG. 3, this embodiment provides an orthopedic surgical robot comprising the aforementioned automatic screw-placement system and a robotic system. The robotic system includes a master control cart 62 and a main unit cart 63. The master control cart 62 includes an industrial computer 64, a display 65, and an optical camera 66. The main unit cart 63 includes a robotic arm 67, whose distal end comprises a first connecting structure for connecting the power platform 69. The outer surface of the first connecting structure is equipped with an optical marker module 68, which is configured to identify the pose of the robotic arm.

Further, the screw-insertion module 3 includes the robotic arm interface 8. The screw-insertion module 3 is connected to the distal end of the robotic arm 67 through the robotic arm interface 8, thereby linking the power platform 69 to the orthopedic surgical robot. The orthopedic robot can then control the screw-insertion process of the automatic screw-placement system via commands, thereby realizing the automated insertion of the pedicle screw 7.

It is understood that, in this embodiment, by mounting the power platform 69 on the distal end of the robotic arm 67, the entire process from the insertion of the Kirschner wire 1 to the insertion of the pedicle screw 7 can be performed automatically, greatly reducing manual operation by the surgeon.

Referring to FIG. 2, in the orthopedic surgical robot of this embodiment, each component of the robotic system (industrial computer 64, display 65, optical camera 66, and optical marker module 68) has specific functions. Specifically, the industrial computer 64 serves as the control center of the robotic system, processing data from various sensors and sending instructions to the robotic arm and other actuators. It can process large amounts of real-time image, position, and pose information and store the data for subsequent analysis or record-keeping. Surgeons can perform planning, recognition, and real-time monitoring through a keyboard and mouse. The master control sends commands to the robotic arm via network cable to execute various operations. The industrial computer 64 runs various control algorithms and image-processing algorithms for path planning, pose recognition, and error correction. The display 65 provides an intuitive interface for the operator, allowing real-time observation of the robot's working status, comprising the position and pose of the robotic arm and the surgical area images. The display 65 is usually equipped with touch functionality, allowing operators to set parameters and make manual adjustments directly on the interface. The optical camera 66 captures images of the surgical area, providing high-resolution visual feedback. Through image processing, the optical camera 66 can accurately locate target positions and track movement in real time. The optical marker module 68, mounted on the distal end of the robotic arm, identifies the pose (position and orientation) of the robotic arm. It usually consists of a set of reflective markers, which can be tracked by external cameras or other optical sensors. Based on the recognized pose information, the optical marker module 68 helps the robotic arm achieve precise motion control, ensuring movement along the planned trajectory.

Further, the industrial computer, display, optical camera, and optical marker module work together to form a closed-loop control system. The industrial computer handles overall control and data processing, the display provides the human-machine interface, the optical camera provides visual feedback, and the optical marker module ensures precise operation of the robotic arm. This significantly improves overall accuracy and reliability, allowing the automatic screw-placement system to operate efficiently in complex medical environments. Real-time monitoring and feedback not only increase surgical success rates but also reduce patient trauma and recovery time, improving overall surgical efficiency.

Referring to FIG. 2, the working process of the orthopedic surgical robot in this embodiment can be illustrated with the following steps:
Step 1: After preparing the operating table, place the patient in the prone position.
Step 2: Place the patient tracker 70 on the patient's vertebra and use the optical camera 66 to identify the position of the vertebra.
Step 3: Appropriately position the main unit cart 63 and master control cart 62. Typically, the master control cart 62 is located at the patient's head side, and the main unit cart 63 is placed on the opposite side near the surgical site (i.e., the vertebra for screw placement). Cover both carts with sterile drapes to maintain a sterile environment.
Step 4: Use the optical camera 66 to capture intraoperative CBCT images and transmit them to the industrial computer 64 on the master control cart. Plan the implantation position and specifications of the pedicle screw 7 as needed.
Step 5: Fix the sterilized power platform 69 to the distal end of the robotic arm 67, rigidly connecting it with the optical marker module 68.
Step 6: After securing the pedicle screw with screws, it can be quickly connected to the power platform 69. In minimally invasive procedures, a protective sleeve is provided to shield the pedicle screw 7, preventing contact with soft tissue during skin entry and screw placement. The form of this sleeve is not limited.
Step 7: Load the Kirschner wire 1 into the power platform 69 and align its tip 6 with the tip of the pedicle screw 7, then tighten.
Step 8: Following the planned trajectory, the robotic system guides the robotic arm to the screw placement position, aligning the axes of the pedicle screw 7 and power platform 69 with the planned screw axis, and inserts the pedicle screw 7 along the axis into the prepared skin incision. Stop when the tip of the pedicle screw 7 contacts the cortical bone.
Step 9: The power platform 69 controls the pedicle screw 7 to rotate at high reverse speed (>10,000 RPM) and advance 2-5 mm along the axis to penetrate the cortical bone. A protective sleeve between the pedicle screw 7, screw-mounting module 10, and the skin prevents soft tissue abrasion.
Step 10: The power platform 69 controls the Kirschner wire 1 to advance 5-10 mm beyond the pedicle screw 7 tip, forming a guide within the pedicle.
Step 11: The power platform 69 controls the pedicle screw 7 to rotate forward while applying axial pressure. The pedicle screw 7 engages with bone under the guidance of the Kirschner wire 1, gradually entering the bone until the tips of the pedicle screw 7 and Kirschner wire 1 align.
Step 12: Repeat steps 10 and 11 until the pedicle screw 7 reaches the planned position. The display 65 allows monitoring of whether the planned position is achieved.
Step 13: After the power platform 69 controls Kirschner wire 1 to retract to a certain position, manually release the screw-mounting module 10. The robotic arm retracts the power platform 69, completing the implantation of one pedicle screw.

It should be noted that this embodiment provides an orthopedic surgical robot comprising the robotic system, automatic screw-placement system, and corresponding Kirschner wire and pedicle screw. The power platform is fixedly connected to the distal end of the robotic arm and rigidly connected to the optical marker module. Through the coordinated design of the pin-insertion module and screw-insertion module, the system integrates Kirschner wire positioning and screw implantation onto the same robotic arm platform. With the robotic system's command control, the system significantly shortens surgical time, reduces contamination risk, solves technical problems in the prior art (such as Kirschner wire slippage and manual screw implantation), and achieves fully automated and precise control from Kirschner wire positioning to screw insertion, providing a more efficient, stable, and safe solution for robot-assisted spinal surgery.

The technical features described above can be combined arbitrarily. Although not all possible combinations are described, any combination of these technical features should be considered encompassed by this description, provided that such combinations do not conflict.

The specific embodiments of the present disclosure described above do not limit the scope of protection of the disclosure. Any other modifications and variations made based on the technical concept of the present disclosure should be included within the scope of the claims of the disclosure.

What is claimed is:

1. An automatic screw-placement system, comprising:
a Kirschner wire (1), comprising a needle body (4) and a needle head (5), the needle body (4) being fixedly connected to the needle head (5), the needle head (5) comprising a needle tip (6), the needle tip (6) being located at an end of the needle head (5);
a pedicle screw (7), comprising a screw body (30) and a milling assembly (31); and
a power platform (69), comprising a pin-insertion module (2) and a screw-insertion module (3), the pin-insertion module (2) being configured to insert the Kirschner wire (1) into patient's spine, and the screw-insertion module (3) being configured to insert the pedicle screw (7) into a pedicle of the patient's spine.

2. The automatic screw-placement system according to claim 1, wherein the pin-insertion module (2) is detachably connected to the Kirschner wire (1), the screw-insertion module (3) comprises a robotic-arm interface (8), and the robotic-arm interface (8) is configured to connect a robotic arm (67).

3. The automatic screw-placement system according to claim 2, wherein the power platform (69) further comprises a screw-mounting module (10), and the pedicle screw (7) is mounted on the screw-mounting module (10);
the screw-insertion module (3) is fixedly connected to the pin-insertion module (2), and the screw-mounting module (10) is detachably connected to the screw-insertion module (3).

4. The automatic screw-placement system according to claim 1, wherein a diameter of the needle body (4) of the Kirschner wire (1) is greater than a diameter of the needle tip (5), the end of the needle tip (5) includes a double-sided cutting edge (12) and a chip-removal groove (13), the double-sided cutting edge (12) is configured to cut bone, and the chip-removal groove (13) is configured to remove debris.

5. The automatic screw-placement system according to claim 2, wherein the pin-insertion module (2) comprises a transmission shaft (14), the screw-insertion module (3) comprises a screw-insertion housing (15), and a first motor (16) and a main gear (17) are disposed inside the screw-insertion housing (15); the first motor (16) is connected to the main gear (17), the main gear (17) is connected to the transmission shaft (14), and the first motor (16) is configured to drive the main gear (17) to rotate and thereby rotate the transmission shaft (14).

6. The automatic screw-placement system according to claim 5, wherein a second motor (18) and a gear shaft (19) are further disposed inside the screw-insertion housing (15), the second motor (18) is connected to the gear shaft (19), the second motor (18) is configured to drive the gear shaft (19) to rotate, and an inner sleeve (20) is disposed inside the second motor (18) for allowing the Kirschner wire (1) to pass through.

7. The automatic screw-placement system according to claim 6, wherein a third motor (21), a reducer (22), and a clutch (23) are further disposed inside the screw-insertion housing (15); the third motor (21) is connected to the reducer (22), the reducer (22) is connected to the clutch (23), one end of the gear shaft (19) is provided with a driving gear (24), the driving gear (24) is connected to the clutch (23), and an other end of the gear shaft (19) is connected to a first connecting device (25), and the first connecting device (25) is detachably connected to the screw-mounting module (10).

8. The automatic screw-placement system according to claim 7, wherein the pin-insertion module (2) comprises a pin-insertion housing (26), and an upper sleeve (27) and a fixing sleeve (28) are disposed inside the pin-insertion housing; the upper sleeve (27) and the fixing sleeve (28) are fixedly connected, internal spaces of the upper sleeve (27) and the fixing sleeve (28) communicate with each other for allowing the Kirschner wire (1) to pass through, and the fixing sleeve (28) is fixedly connected to the transmission shaft (14).

9. The automatic screw-placement system according to claim 3, wherein the power platform (69) further comprises a retaining frame (29), the retaining frame (29) is fixedly connected to the screw-insertion module (3), and the retaining frame (29) is detachably connected to the screw-mounting module (10).

10. The automatic screw-placement system according to claim 1, wherein a movement space (33) extending along an extension direction of the screw body (30) is defined inside the screw body (30); the movement space (33) is configured for the Kirschner wire (1) to pass through; the milling assembly (31) is mounted at an end of the screw body (30), threads are defined on a surface of the milling assembly (31), and grooves are formed on the threads; the milling assembly (31) comprises at least two cutting edges, and the at least two cutting edges are circumferentially spaced apart at the end of the screw body (30).

11. The automatic screw-placement system according to claim 10, wherein the pedicle screw (7) further comprises a screw head (34), the screw head (34) is disposed at an end of the screw body (30) away from the milling assembly (31); a mounting hole is formed in the screw head (34), and the mounting hole communicates with the movement space (33).

12. The automatic screw-placement system according to claim 11, wherein the pedicle screw (7) further comprises a screw-body long tail (36), and the screw-body long tail (36) is fixedly connected to the screw head (34);
the screw-body long tail (36) has a connecting end (37) and a fixing end (38), a first mounting space (39) is defined in the connecting end (37) of the screw-body long tail (36), a second mounting space (40) is defined in the fixing end (38) of the screw-body long tail (36), the first mounting space (39) is communicated with the second mounting space (40), and the screw head (34) is movably disposed inside the first mounting space (39).

13. The automatic screw-placement system according to claim 12, wherein the pedicle screw (7) further comprises a connecting assembly (41), the connecting assembly (41) comprises: a first connecting portion (42) and a second connecting portion (43) connected in sequence, the first connecting portion (42) and the second connecting portion (43) both extend in the extension direction of the screw body (30), the first connecting portion (42) is connected to the screw-mounting module (10), the second connecting portion (43) is rotatably disposed inside the second mounting space (40), and the second connecting portion (43) is rotatably connected to the fixing end (38) of the screw-body long tail (36).

14. An orthopedic surgical robot, comprising:
the automatic screw-placement systems according to claim 1;
a robot system, comprising a main control cart (62) and a main machine cart (63), the main control cart (62) comprising an industrial computer (64), a display (65), and an optical camera (66), the main machine cart (63) comprising a robotic arm (67), an end of the robotic arm (67) comprising a first connecting structure configured to connect the power platform (69); and
an optical marker module (68) fixedly disposed on an outer surface of the first connecting structure, and configured to identify a pose of the robotic arm (67).
